(19) United States
(12) Patent Application Publication
(10) Pub. No.: US 2012/0095351 A1
(43) Pub. Date: Apr. 19, 2012

(54) BLOOD TREATMENT DEVICE

(75) Inventors: Hans-Peter Klose, Stuttgart (DE); Bernd Huber, Korntal (DE)

(73) Assignee: ROBERT BOSCH GMBH, Stuttgart (DE)

(21) Appl. No.: 13/260,498

(22) PCT Filed: Jan. 29, 2010
(86) PCT No.: PCT/EP2010/051056

§ 371 Date: Dec. 21, 2011

(30) Foreign Application Priority Data
Mar. 26, 2009 (DE) 10 2009 001 901.4

(51) Int. Cl. A61B 5/0205; A61B 5/021

(52) U.S. Cl. 600/483; 600/485

(57) ABSTRACT

In a device for treating blood in an extracorporeal circuit, at least one blood pump and at least one filtering device are provided for carrying out the blood treatment. The device includes at least one pressure-measuring element for detecting the blood pressure, which is characterized in that the pressure-measuring element has a sensor unit and an RFID transponder unit.

### FIELD OF THE INVENTION

The present invention relates to a device for the treatment of blood in an extracorporeal circuit, the device having at least one blood pump, at least one filtering device, and at least one pressure-measuring element for detecting the blood pressure.

### BACKGROUND INFORMATION

With kidney failure or limited kidney function, blood treatment, especially blood purification, is required in order to compensate for the limited or lacking function of the kidneys, or to replace it. In so doing, filtering of the blood via a semipermeable membrane is performed, in which substances from the patient's blood enter the so-called dialysis fluid by way of dialysis. The exchange usually takes place in an extracorporeal circuit. For this purpose the dialysis fluid and the blood of the patient are guided past each other in a filtering device having a semipermeable membrane, according to the counter-current principle, the substance exchange taking place in the process. This is called hemodialysis.

For the extracorporeal blood circulation in a dialysis operation, blood is first

removed from the patient via an arterial access point. A blood pump transports and forwards the blood to a filtering device, where the actual dialysis is performed via the semipermeable membrane. An access point for medication, e.g., the addition of Heparin to reduce blood clotting, is usually provided between the blood pump disposed upstream from the filtering device, and the actual filtering device. Possible air bubbles are removed in a so-called air bubble trap downstream from the filtering device, before the purified blood is returned to the patient via a venous access.

In addition to hemodialysis, hemofiltration is known as well, in which water is removed from the blood via a membrane, using pressure, and replaced by an electrolyte fluid. Hemodialysis and hemofiltration also may be used in combination. Another blood treatment method is hemoperfusion, which may be used in acute poisoning cases, in particular. In this case the blood is pumped through adsorbent substances (e.g., activated charcoal) in order rid the blood of certain substances such as overdosed medications.

Since complications may arise during dialysis and other blood treatment methods, the devices must have sufficient monitoring functions in order to minimize the risk for the patients and in order to allow counter measures to be initiated if necessary. Of special importance in this context is a drop in the blood pressure, which frequently requires the dialysis treatment to be stopped or at least calls for an intervention in the treatment. For this reason devices for measuring the blood pressure are frequently provided at different locations of the extracorporeal blood circuit. The arterial blood pressure is typically measured upstream from the blood pump. The venous blood pressure is measured downstream from the filtering device. Furthermore, the blood pressure is able to be detected upstream from the filtering device, e.g., downstream from an adding point for medication.

As a rule, the pressure-measuring cells for detecting the blood pressure are permanently installed in the actual dialysis device. The respective points of the blood-carrying tubes are connected to the pressure sensors in the dialysis device in order to detect the blood pressure. This is done hydrostatically, for example, using a so-called press-fit coupling with air buffer, or via a tap line of the tubes. This coupling of the tube systems to the permanently installed pressure sensors poses a number of inherent problems. The connections may be faulty and thus lead to leaks, for example. This could falsify the measuring result and, in particular, also allow air to enter the blood circuit, which causes the known serious risks for the patient. Furthermore, the sterility of the connections must be ensured. In addition, the branching of the lines increases the clotting risk of the blood.

This calls for higher doses of anti-coagulants. The branching of the lines requires a greater extracorporeal blood volume. The risk caused by a faulty operation, especially during dialysis at home, and the risk of faulty displays rise as well. In addition, such a blood pressure measurement entails considerable manual work in connection with the device.

Especially in cases of chronic renal disease, dialysis in a home setting in the form of so-called home dialysis is endeavored. This requires uncomplicated handling for the patient or for persons tending to the patient, who may possibly not have received comprehensive medical training. The mentioned disadvantages, which may also arise in the application by medically trained personnel, are thus also a factor in home dialysis, in particular.

### SUMMARY

The blood treatment device according to example embodiments of the present invention and the use of an RFID transponder unit and a sensor unit as described below remedy the described disadvantages of conventional systems and provide a dialysis or blood treatment device that allows the blood pressure to be measured and/or other medical parameters to be detected during the treatment in an especially advantageous manner.

The device according may be used for treating blood in an extracorporeal circuit such as for hemodialysis, hemofiltration or hemoperfusion. In this context, the blood is guided in an extracorporeal circuit having at least one blood pump, and passes through a filtering device, where the actual blood treatment takes place, especially the osmotic substance exchange through a semipermeable membrane. The filtering device is a conventional fibrilla filter, for example, which conventionally is used for hemodialysis. Furthermore, the device is equipped with at least one pressure-measuring element for detecting the blood pressure in order to record changes in the blood pressure, which may indicate complications in performing the dialysis treatment. The device is characterized by the fact that the pressure-measuring element has a sensor unit for detecting the blood pressure and an RFID transponder unit, which allows a wireless transmission of the measured values to an external device as receiver or as reading device. This receiver preferably is part of the device, e.g., the dialysis device. In other specific embodiments, the receiver may be disposed externally, e.g., on or in a separate monitoring computer.

The present device utilizes the known RFID technology. RFID is the abbreviation of the English term “Radio Frequency Identification”. This technology is usually employed for the automatic identification of objects and living things, especially humans and animals. Even an automatic detection and storage of data has since become possible with the aid of this technology. The RFID system includes a transponder unit and an external device, especially a reading device. The reading device generates an electromagnetic high-frequency field, via which a data transmission from transponder to reading device takes place. Radio frequencies in the radio frequency range of a few 100 MHz to 100 KHz are typically used; in the meantime, even frequencies up to the microwave range are utilized as well. The data transmission thus takes place in a contactless manner. In addition to the data transmission between the transponder and the external receiver, the RFID system also allows data to be written to the transponder, as the case may be. For instance, the use of an RFID transponder unit as component of a pressure-measuring element in a device as described herein has the advantage, among other things, that no physical connection of the measuring element to the dialysis device as required in conventional blood pressure measurements is necessary. Instead, the blood pressure measurement may take place wirelessly, so that the handling is made considerably easier for the medical personnel or caretakers, or also for the patient himself, since no separate connections are required for the pressure-measuring sensors. The coupling of the pressure-measuring sensors to the actual dialysis device very advantageously takes place by means of a wireless and also contactless data exchange. Mechanical or hydrostatic coupling is omitted, which simplifies the handling and makes corresponding coupler elements superfluous.

A particular advantage of the device is that the used sensors are able to be detected via unambiguous sensor identification. This is advantageous in particular in cases where multiple RFID-capable dialysis devices are placed in close proximity to each other, e.g., in dialysis centers; suitable, collision-encumbered RFID transmission protocols make it possible for the reader device to receive and analyze the correct measuring data, without any risk of mix-ups of patent data, for example. In an advantageous manner, a sensor which detects mechanical stresses in a vessel wall, e.g., the tube wall, may be used as sensor unit for the blood pressure measurement, this sensor unit detecting a change in the wall characteristics of the tube wall through a change in the wall tension. Suitable blood pressure sensors are known from German Published Patent Application No. 10 2007 038 402, for example, which describes an implantable RFID blood pressure sensor. However, these pressure-measuring sensors have the disadvantage that the blood pressure is detected indirectly, by measuring pressure-dependent material or construction parameters in the pressure-carrying line, which makes the pressure measurement also dependent on material parameters of the wall material, such as elasticity, pressure module, etc. In an example embodiment, a sensor unit which performs a pressure measurement directly in the blood, is therefore used in order to be able to thereby eliminate other fault sources. Here, the placement of the sensor unit is not restricted to blood-carrying lines or tubes; instead, the sensor unit may be placed at any point, e.g., directly inside a fibrilla filter.

Preferably, the pressure-measuring element(s) is/are integrated into a tube, a tube connection, into the filtering device, and/or into a filter connection. The sensor unit and the RFID transponder unit may be kept very small in their dimensions, so that an integration at the various points in the extracorporeal blood circuit is readily possible, without any real adverse effect on the blood flow. This advantage plays a role especially when passive transponders are used in the RFID transponder unit. The energy supply of passive transponders is implemented via the external receiver, so that passive transponders may have a very compact and small design and, in particular, need not have their own energy supply. In this manner the transponder does not require maintenance.

The pressure-measuring elements preferably may be used in the usual locations for the blood-pressure measurement in the extracorporeal circuit of a device; for an arterial pressure measurement, this preferably is upstream from the blood pump, and for the venous pressure measurement, preferably downstream from the filtering device and upstream from an air bubble trap. Furthermore, a pressure-measuring element, for instance, may be used directly in front of the filtering device, either in addition or as an alternative. Since the pressure-measuring elements do not require any further installation, a great advantage of the present invention is that a plurality of pressure-measuring elements is able to be used without special effort on the part of the operators or the patient.

In an example embodiment, the RFID transponder unit, and preferably the entire pressure-measuring element, is a single-use unit. The pressure-measuring element including the sensor unit and the RFID transponder unit is intended for single use. Suitable single-use RFID transponder units are already known. They have the advantage of being usable in conjunction with other single-use components in the treatment of blood. For example, tube sets and filtering units which are provided in sterile form and disposed of after use are currently already the rule. Cleaning, reconditioning and sterilizing such units frequently does not make sense for cost reasons. Therefore, the pressure-measuring element is likewise provided as single-use component, so that, for example, the correspondingly equipped tube set is connected to the connections of the filtering device and to corresponding access points on the patient in the known manner, without any further installation being required for the blood-pressure measurement. Furthermore, it is possible that only the transponder unit is provided for single use, whereas the sensor unit is reusable.

In an especially advantageous manner, the device has further measuring elements which include a sensor unit and an RFID transponder unit in each case, the further measuring elements detecting additional medical parameters. These additional measuring elements may be provided to record, for example, the blood flow rate and the temperature, and/or to detect gas inclusions, especially air bubbles. In a hemodialysis or in other blood treatment methods having an extracorporeal blood circuit, thorough monitoring of various parameters is required in order to avoid complications or to detect them in a timely manner. The wireless coupling of appropriate sensors is thus especially advantageous for these additional parameters as well, since the conventional measurement of these additional parameters may produce potential error sources, especially in connection with home dialysis, such as by additional tube connections.

In addition, example embodiments of the present invention include the use of an RFID transponder unit and a sensor unit as measuring element for the purpose of detecting medical parameters, especially the blood pressure, the blood flow rate, the temperature, and/or gas inclusions, in a device for treating blood in an extracorporeal circuit. Additional medical parameters in this context are, for example, blood sugar values or certain gas concentrations in the blood. The device includes at least one blood pump and at least one filtering device and may be provided for hemodialysis or hemofiltration, for example. More specifically, this is a hemodialysis device which is equipped with a receiver for reading out data of one or especially a plurality of RFID transponder unit(s). The RFID transponder units are integrated into the system of the extracorporeal blood circuit together with appropriate sensor units, e.g., as pressure-measuring elements, for instance into the corresponding tube sets or into the filtering device. With regard to additional features and advantages of this use, reference is made to the above description.

One particular advantage of the device and the use is that the RFID transponder unit of the pressure-measuring element or some other measuring element offers additional possibilities in connection with the data storage on the RFID transponder.

The use of an RFID transponder unit in the described pressure-measuring element allows measured values and/or patient data to be documented in an especially advantageous manner. For example, the measured values regarding blood pressure may be recorded as characteristic of the blood pressure over the treatment period and stored, or they may continue to be documented and analyzed following the treatment. When using an RFID transponder unit, the treatment documentation, especially the blood pressure characteristic, and also other relevant data may be carried out in automated and optimized manner.

The measured values and/or the patient data may be forwarded telemetrically, so that, for example, monitoring or an analysis of the treatment may take place from a remote location. For example, the dialysis treatment may be carried out as home dialysis in the home environment of the patient, and the data of the blood pressure and possibly additional data may be transmitted, via the transponder units, to the dialysis device where a reader is integrated; from here, the data are transmittable via a telephone line or a corresponding data line, for instance, possibly together with other information about the patient, to a central location such as a dialysis center or a medical office, where the data are documented and taken into account in the further treatment of the patient.

In an especially advantageous manner, the use for detecting malfunctions is possible. For example, an alarm may sound if a drop in blood pressure below a predefinable threshold is detected. Preferably, the use is provided for initiating one or more emergency measures when a malfunction is detected; for example, if suitable threshold values are stored on the transponder or a control unit, the termination of the dialysis treatment may be initiated as soon as a predefinable threshold of the blood pressure is not met. At critical measured blood pressure values during a dialysis treatment, an alarm is generally triggered at the device, so that a caretaker can approach the patient and ascertain the reason for the alarm. When using an RFID transponder unit, the integration of the blood pressure values in a medical data system makes it possible for the system itself to immediately propose a particular action in response to the detection of critical measured values, especially in conjunction with additional measured data, the patient history and possibly corresponding expert systems. In this way, possibly life-saving measures are able to be initiated much faster, so that the risks for the patient are reduced.

The RFID transponder unit may be employed for component detection and/or identification. Due to the storage of data on the RFID transponder, an identification of the corresponding elements of the system is able to be provided. For example, when a pressure-measuring element is situated within the filtering device, the filtering component may be assigned an identification. The same applies to the placement of a pressure-measuring element in a tube set, e.g., a tube set for the arterial component or for the venous component of the extracorporeal blood circuit. This makes it possible to check whether the correct filter and/or the correct tube set were/was used with respect to the device or with respect to the patient. Furthermore, the detection of new components, protection against counterfeits, a compatibility check and/or protection against re-use are possible in this manner. For instance, the work sequence in dialysis centers may be optimized in that the stock is able to be administered via the component detection and/or identification, and orders are able to be implemented in automated manner when the supplies drop below a specifiable value.

Over all, the use of an RFID transponder unit in conjunction with a sensor unit, e.g., as integrated component in a single-use tube set or a filtering device for the hemodialysis, considerably simplifies the operation with regard to the structure and setup of the dialysis system on the part of the patient. The RFID transponder unit ensures an exchange protection of the utilized units, protection against incorrect use, counterfeit protection, protection against reuse, or protection against the use past a minimum use date. Furthermore, the monitoring of the patient is able to be facilitated via the RFID transponder unit, for instance in telemedical applications, where not only the actual measuring data are able to be transmitted, but other patient data as well. These advantages are important especially in home hemodialysis, which is performed by the patient himself or by a family member or caretaker in the home environment. The handling and the setup of the blood treatment device is simplified considerably by the RFID transponder unit that is utilized, and operator mistakes are avoided, especially since practically no prior medical knowledge is required for use in the home environment. For example, the problem of non-sterile or leaky connections with the attendant consequences for the health and life of the patient is solved in this manner.

Even when the device is used and set up by medically trained personnel, e.g., in a dialysis center, the advantages of the system play a role. For example, the setup time per treatment is reduced considerably. Calibrations of the pressure-measuring elements can be carried out in automated manner. No sterilization of the conventionally required connections for the blood-pressure measurement is required. This avoids error sources, and the time requirement for the technical installation of the hemodialysis device is lower. The caretaking expense per patient for the technical installation alone is less, so that this time may be used for other purposes. Manual documentation is dispensed with; the data, especially the measured data for the blood pressure, may be archived in automated manner. A telemedical analysis of the data facilitates patient monitoring. For example, several patients are monitorable at a central station. In the event of malfunctions and, in particular, in cases where the blood pressure drops below specifiable thresholds, the required measures may be initiated very rapidly, possibly in automated manner.

Further features and advantages of example embodiments of the present invention are described below with reference to the FIgures. The individual features may be realized by themselves or in combination with each other.

### BRIEF DESCRIPTION OF THE DRAWINGS

FIG. 1 a schematic illustration of the extracorporeal blood circuit when performing a hemodialysis using a device according to an example embodiment of the present invention;

FIG. 2 a schematic sectional view of a pressure-measuring element to record the blood pressure, disposed on a tube, and

FIG. 3 (A) a schematic illustration of the system of pressure-measuring elements in a filtering device, and (B) a detail view of the pressure-measuring element in a sectional view.

### DETAILED DESCRIPTION

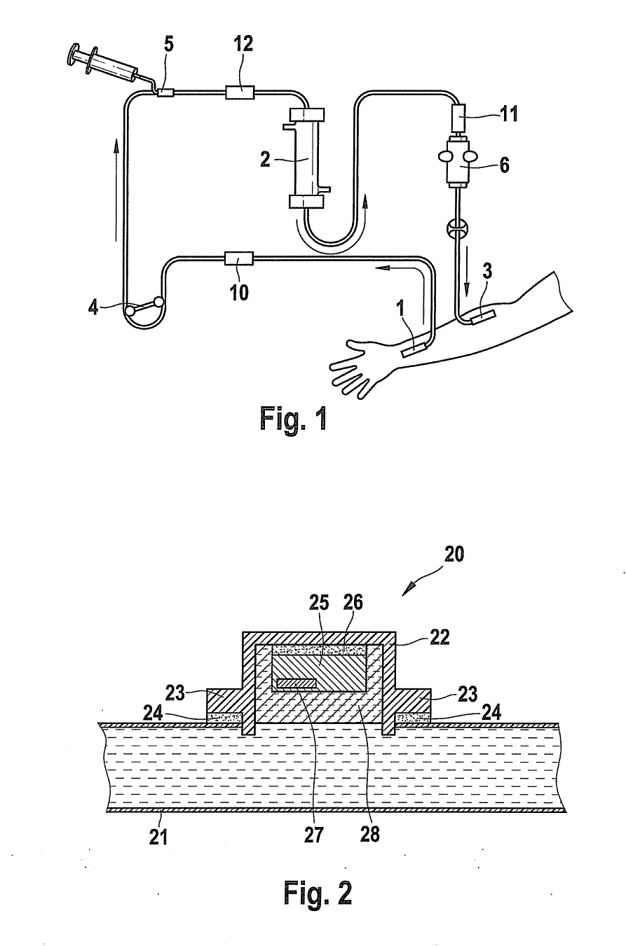

FIG. 1 shows the extracorporeal blood circuit during hemodialysis. This blood circuit is subdivided into an arterial tube system between arterial blood withdrawal location 1 and filtering device 2. The actual dialysis of the blood takes place in filtering device 2, via a semipermeable membrane, the substance exchange between blood and dialysis fluid (not shown) taking place by osmosis. The venous tube system is situated between filtering device 2 and a venous access point 3 to the patient. The external blood circulation is driven via blood pump 4. An adding point 5 for medication such as Heparin to reduce blood coagulation is situated between blood pump 4 and filtering device 2. Prior to returning the blood to the patient again via venous access point 3, it runs through an air bubble trap 6. A blood-pressure measuring element 10 is disposed in the arterial tube system upstream from blood pump 4. A blood-pressure measuring element 11 for the venous pressure measurement is provided upstream from air bubble trap 6. Furthermore, another blood-pressure measuring element 12 may be disposed upstream from filtering device 2. These various blood-pressure measuring elements 10, 11, 12 include a blood-pressure sensor unit and an RFID transponder unit. This dispenses with a hydrostatic or mechanical coupling of the measuring units to the tube system. The detection and forwarding of the blood pressure takes place electronically and in contactless manner. The blood-pressure measuring elements have a small and compact design; the transponder unit preferably is a passive transponder unit whose energy supply is implemented via a receiver (not shown), which is integrated into the dialysis device, for example. The pressure-measuring elements are integrated in the different tubes and connections and do not require further installation. These are preferably single-use units, which are utilized together with the already common single-use tube sets or single-use filtering devices, and are disposed of after use. As an alternative or in addition to the pressure-measuring elements shown here, additional measuring elements for recording other medical parameters may be provided, which likewise include an RFID transponder unit.

Because of the possibility of data storing and data writing offered by the transponder unit, the use of the device according to an example embodiment of the present invention allows measured values and/or patient data, for instance, to be documented in automated fashion and thus in a very simple manner for the operators. Furthermore, the RFID transponder unit may be used for detecting malfunctions and for triggering emergency measures by communicating with corresponding control units. Finally, the RFID transponder unit may be used in an especially advantageous manner for component detection and/or identification if the pressure-measuring element including the RFID transponder unit is integrated into a tube set or a filtering device, for instance, and the transponder unit is provided with the corresponding data that allow component detection and/or identification.

FIG. 2 shows a schematic sectional view of a pressure-measuring element 20, which is disposed on a tube 21 as component of an extracorporeal circulation system of a dialysis device. Pressure-measuring element 20 includes a housing 22, which is mounted on tube 21 by means of an adhesive layer 24 using projections 23. Pressure sensor 25, which is fixed in place in the interior of housing 22 with the aid of an adhesive layer 26, is provided within housing 22. An RFID transponder unit 27 is integrated into sensor 25. Sensor 25 is embedded in a bio-compatible gel 28 inside housing 22. Sensor 25 is suitable for a direct pressure measurement and is in direct connection with the fluid or the blood inside tube 21 via biocompatible gel 28. Gel 28 transmits the pressure to be detected to sensor 25. Such a pressure-measuring element 20 may be integrated directly into a tube wall, for example.

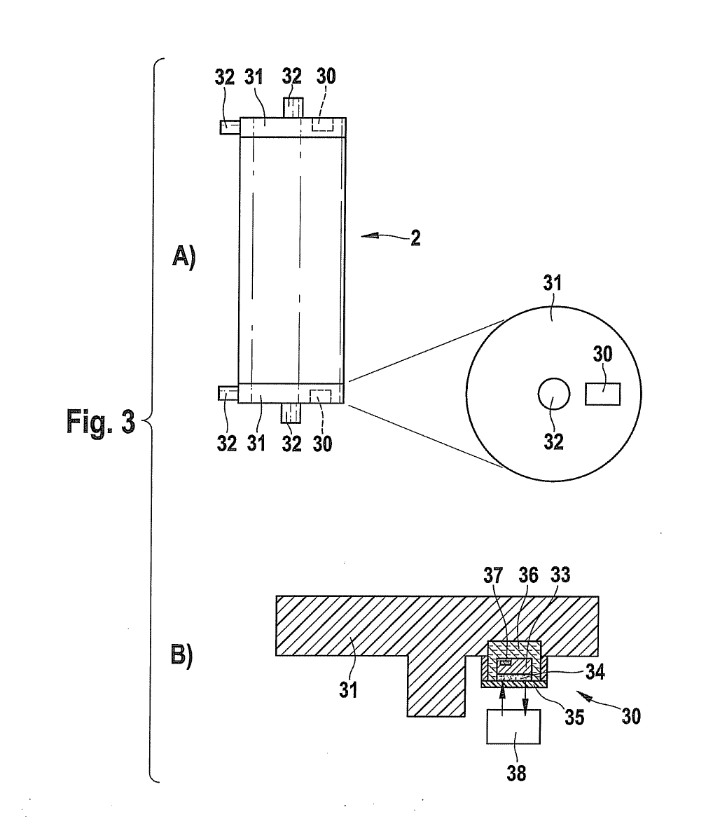

Another option for the placement of pressure-measuring elements 30 within an extracorporeal circuit of a dialysis device can be gathered from FIG. 3. FIG. 3 (A) shows the placement of pressure-measuring elements 30 in tops 31 of a filtering device 2, e.g., a fibrilla filter. Pressure-measuring elements 30 are situated in the region of connections 32 for the blood and the dialysis fluid. FIG. 3 (B) shows the placement of pressure-measuring element 30 in detail. Via an adhesive layer 34, sensor 33 is disposed within a housing 35, in top 31 of filtering device 2. Sensor 33 is embedded in a bio-compatible gel 36, via which sensor 33 is in direct contact with the fluid or the blood for the direct pressure measurement. An RFID transponder unit 37 is integrated into sensor 33. Depending on the development of the sensor, the measured values of the blood pressure and other biological parameters detected by sensor unit 33 are wirelessly transmitted to an RFID receiver or to a reading device 38, via RFID transponder unit 37. The transponder in RFID transponder unit 37 preferably is a passive transponder, whose energy supply is implemented via external receiver or reading device 38. Top 31 with housing 35 may be produced as injection-molded part, for instance, into which the sensor element together with the jelly is introduced by pressing it into a corresponding depression of the housing, for instance.

1-12. (canceled)

13. A device for treatment of blood in an extracorporeal circuit, comprising:
at least one blood pump;
at least one filter device; and
at least one pressure-measurement device adapted to detect blood pressure, the pressure-measurement device including a sensor unit and an RFID transponder unit.

14. The device according to claim 13, wherein the pressure-measurement device is integrated into at least one of (a) a tube, (b) a tube connection, (c) the filtering device, and (d) a filter connection.

15. The device according to claim 13, wherein the RFID transponder unit includes a passive transponder.

16. The device according to claim 13, wherein the pressure-measurement device is adapted for at least one of (a) arterial pressure measurement and (b) venous pressure measurement (11).

17. The device according to claim 13, wherein the RFID transponder unit is arranged as a single-use unit.

18. The device according to claim 13, further comprising additional measurement units adapted to detect additional parameters, the additional measurement units each having a sensor unit and an RFID transponder unit.

19. The device according to claim 18, wherein the additional parameters include at least one of (a) blood flow rate, (b) temperature, and (c) gas inclusions.

20. A system, comprising:
a device adapted to treat blood in an extracorporeal circuit, the device including at least one blood pump and at least one filter device; and
a measurement device, including an RFID transponder unit and a sensor unit, adapted to detect medical parameters.

21. The system according to claim 20, wherein the medical parameters include at least one of (a) blood pressure, (b) blood flow rate, (c) temperature, and (d) gas inclusions.

22. The system according to claim 20, wherein the system is adapted to document at least one of (a) measured values and (b) patient data.

23. The system according to claim 20, wherein the system is adapted for telemetric forwarding of at least one of (a) measured values and (b) patient data.

24. The system according to claim 20, wherein the system is adapted to detect malfunctions.

25. The system according to claim 20, wherein the system is adapted to trigger emergency measures.

26. The system according to claim 20, wherein the system is adapted for at least one of (a) component detection and (b) component identification.
